(19) United States
(12) Patent Application Publication
(10) Pub. No.: US 2003/0017507 A1
(43) Pub. Date: Jan. 23, 2003

(54) Relative protein quantitation phosphoproteins using stable isotope labeling

(76) Inventor: Richard S. Johnson, Seattle, WA (US)

Correspondence Address: IMMUNEX CORPORATION, LAW DEPARTMENT, 51 UNIVERSITY STREET, SEATTLE, WA, 98101

(21) Appl. No.: 10/160,616

(22) Filed: May 30, 2002

Related U.S. Application Data
(60) Provisional application No. 60/294,803, filed on May 30, 2001.

(51) Int. Cl. G01N 33/53; C07K 14/435

(52) U.S. Cl. 435/7.5; 530/404

(57) ABSTRACT

In one aspect, the present invention provides a method for incorporating a stable isotope into a protein or peptide fragment. In the method, a modified protein or peptide fragment is reacted with an agent that includes one or more stable isotopes to provide an isotope-labeled protein or peptide fragment. In another aspect of the invention, methods for measuring protein levels in two protein or peptide fragment mixtures are provided. In these methods, protein levels are measured using stable isotope-coded protein or peptide fragments.

### CROSS REFERENCE TO RELATED APPLICATIONS

This application claims the benefit of priority under 35 U.S.C. §119 to U.S. Provisional Application Serial No. 60/294,803, filed May 30, 2001, the disclosure of which is incorporated herein.

### BACKGROUND OF THE INVENTION

1. Field of the Invention

The present invention is directed to a method for making quantitative protein measurements including relative protein quantitation using stable isotope labeling of phosphoserine-, phosphothreonine-, N-acetyl glucosamine (O-GlcNAc) serine-, and N-acetyl glucosamine threonine-containing proteins and peptides.

2. Description of Related Art

Quantitative proteomics usually involves separation of individual proteins using two dimensional gel electrophoresis (2D-PAGE) and comparison of the staining density. Proteomic analyses using 2D-PAGE can be automated, but only at significant expense requiring automated gel staining and destaining devices, imaging equipment, imaging software, spot cutting robotics, automated in-gel digestion, robotic MALDI plate spotting, and mass spectrometry. Even with an expensive high throughput 2D-PAGE system, one has to be cognizant of the fact that these systems are known to have difficulties with higher molecular weight proteins, membrane proteins, and highly acidic or basic proteins. Despite the high resolution separations of proteins provided by 2D-PAGE, the method still suffers from a limited dynamic range; low abundance proteins are very difficult to detect in the presence of high abundance proteins. Nevertheless, 2D-PAGE has been the state of the art for making quantitative proteomic measurements.

Reversible biotinylation of cysteinyl peptides has been utilized in a method for the rapid identification of components in a protein mixture (Spahr et al., 2000). In a representative method, a protein mixture is digested and the resulting peptide fragment's cysteine residues biotinylated with a cleavable biotinylation reagent (i.e., N-[6-(biotinamido)hexyl]-3′-(2′-pyridyldithio)propionamide, commonly known to as “biotin-HPDP”). The biotinylated peptides are then isolated using avidin affinity chromatography and then eluted from the avidin by treatment with dithiothreitol (DTT), which cleaves the link between the biotin and peptide fragment releasing the peptide fragment. The released peptide fragment has free sulfhydryl groups that are alkylated by treatment with iodoacetamide. The alkylated peptide fragments are then analyzed by LC/MS/MS to provide proteomic information. The method described above simplifies complex peptide mixtures for proteomic analysis.

An alternative to quantitative imaging of 2D-PAGE employs the concept of isotope dilution in the context of proteomic analyses. In such a method, proteolytic peptides are labeled with different stable isotopes depending on the protein source (e.g., control cells versus stimulated cells). Because isotopic labeling of identical peptides will result in nearly equivalent chemical properties, pairs of peptides differing only in the label will elute approximately at the same time and exhibit identical ionization efficiency. The first example of this method was the use of whole cell ¹⁵N labeling to compare wild type and mutant cell lines. This approach is limited to studies of cultured cells, and the isotope coding involves the incorporation of varying numbers of nitrogen atoms in each peptide, hence varying mass differences from peptide to peptide.

Another approach involves N-terminally labeling of proteolytic peptides with isotope-coded nicotinic acid derivatives. This method has a side benefit of directing fragmentation in MS/MS. More recently, whole cell labeling with ¹³C lysine has been shown to be a simple way to introduce a constant mass shift in tryptic peptides.

In addition to the isotope labeling methods noted above, complex protein mixtures have also been quantitatively analyzed using isotope-coded affinity tags and mass spectrometry (Aebersold et al., 1999). The analysis is based on the labeling of a protein's cysteine residues with an isotope-coded affinity tag (ICAT) and subsequent analysis of the tagged protein, or fragment thereof, by mass spectrometry. The ICAT reagents employ cysteine-specific chemical reactivity, an isotope coded linker, and a biotin affinity tag, and introduce a constant mass difference for each cysteine present in the peptide. The ICAT reagent includes a reactive functional group having specificity toward sulfhydryl groups, a biotin affinity tag, and an isotope labeled linker covalently linking the sulfhydryl reactive group with the biotin tag. An advantage of this method is that complex tryptic peptide mixtures can be simplified by the selective isolation of peptides containing cysteine, which is one of the least common amino acids, thus approaching the ideal of obtaining a single peptide per protein.

In a representative method, the cysteinyl residues in a reduced protein sample representing one cell state are derivatized with one isotopic form (e.g., light form, no isotope label) of the ICAT and the equivalent groups in a second cell state are derivatized with another isotopic form (i.e., heavy form, isotope labeled). The two samples are then combined, enzymatically cleaved to produce peptide fragments, and the biotin tagged fragments isolated by avidin affinity chromatography. The isolated fragments are then released and analyzed by microLC-MS/MS. The quantity and sequence identity of the proteins from which the fragments are derived are determined by automated multistage mass spectrometry. Despite the utility of the ICAT method described above, the method requires the use of the relatively sophisticated and expensive ICAT reagent. Furthermore, the mass spectra of tagged protein fragments is obscured by high intensity ions related to the reagent.

The rapid and selective modification of phosphoserine residues for their detection during peptide sequencing has been utilized to overcome the difficulty in unambiguously identifying the sites of serine phosphorylation (M. F. Byford, Biochem. J. 1991, 280, 261-265). In this method, protein phosphoserine residues are treated with dilute alkali in the presence of a Group II metal ion (e.g., barium hydroxide) resulting in β-elimination to provide an α,β-unsaturated residue that is sensitive to nucleophilic addition. Addition of methylamine resulted in the formation of an amino acid residue at the position occupied by the parent phosphoserine residue. The modified residue was readily detected on sequence analysis.

Serine and threonine phosphorylation sites in β-elimination/ethanedithiol addition-modified proteins have been characterized by electrospray tandem mass spectrometry (H. Jaffe et al., Biochemistry 1998, 37, 16211-16224). In this method, phosphoserine and phosphothreonine residues in proteins were converted to Sethylcysteinyl or β-methyl-S-ethylcysteinyl residues and the resulting digest analyzed by mass spectrometry to identify original phosphorylation sites.

Phosphoproteins have also been identified and comparatively quantitated using stable isotope labeling and liquid chromatography/mass spectrometry analysis (W. Weckworth et al., Rapid Commun. Mass Spectrom. 2000, 14, 1677-1681). By this technique, quantification was achieved by β-elimination of phosphate from phosphoserine and phosphothreonine followed by Michael addition of ethanethiol and/or ethane-d₅-thiol selectively at the vinyl moiety of dehydroalanine and dehydroamino-2-butyric acid formed by β-elimination. Protein identification and determination of the phosphorylation sites was made by MS/MS fragmentation.

Enrichment analysis of phosphorylated proteins has been used to probe the phosphoproteome (B. T. Chait et al., Nature Biotechnology 2001, 19, 379-382). In the method, dithioethane was added to modified phosphoserine and phosphothreonine residues by the elimination/addition reaction noted above to provide a product containing a free thiol. The product was subsequently biotinylated and captured by avidin for the purpose of enriching phosphorylated peptides and proteins. The enriched phosphorylated peptides and proteins were analyzed by mass spectrometry.

Despite the advances in protein analysis, there exists a need for rapid and efficient methods for analysis of complex protein mixtures that include phosphoproteins and phosphopeptides. The present invention seeks to fulfill this need and provides further related advantages.

### SUMMARY OF THE INVENTION

In one aspect, the present invention provides a method for incorporating a stable isotope into a protein or peptide fragment. In the method, a protein or peptide fragment is reacted with an agent that includes one or more stable isotopes to provide an isotope-labeled protein or peptide fragment. An α,β-unsaturated residue of the protein or peptide fragment reacts with an isotope-labeled dithiol to provide an isotope-labeled protein or peptide fragment that is a dithiol addition product. In one embodiment, the isotope-labeled protein or peptide fragment is derived from a phosphoserine-containing protein or peptide fragment, phosphothreonine-containing protein or peptide fragment, N-acetyl glucosamine serine-containing protein or peptide fragment, or N-acetyl glucosamine threonine-containing protein or peptide fragment.

By virtue of dithiol addition, the isotope-labeled addition product includes a thioether residue further having an available sulfhydryl group. In an embodiment of the method, the isotope-labeled protein or peptide fragment having an available sulfhydryl group is reacted with an affinity reagent to provide an affinity-labeled protein or peptide fragment that is also isotope labeled. The affinity-labeled protein or peptide fragment can be isolated on a solid phase, and then released from the solid phase to provide an isotope-labeled protein or peptide fragment. In one embodiment, the isotope-labeled protein or peptide fragment includes an available sulfhydryl group that can be alkylated.

In another aspect of the invention, methods for measuring protein levels in two protein or peptide fragment mixtures are provided.

In one embodiment of the method, protein residues capable of β-elimination provide α,β-unsaturated residues in first and second protein mixtures. The α,β-unsaturated residues in the first protein mixture are treated with a first dithiol that includes one or more stable isotopes to provide a first isotope-coded dithiol addition product. The α,β-unsaturated residues in the second protein mixture are treated with a second dithiol that includes no stable isotopes or fewer stable isotopes that the first dithiol to provide a second isotope-coded addition product.

Because the first and second isotope-coded addition products are obtained by reaction with a dithiol, the addition product includes an available sulfhydryl group. In one embodiment, the first and second addition products' available sulfhydryl group are reacted with an affinity reagent to provide first and second affinity-labeled isotope-coded protein mixtures. The first and second affinity-labeled isotope-coded proteins are isolated on a solid phase to provide first and second isolated affinity-labeled isotope-coded proteins. The first and second isolated proteins are released from the solid phase to provide first and second isotope-coded released proteins. The first and second isotope-coded proteins are then analyzed by, for example, mass spectrometry. The first and second protein mixtures can be combined prior to analysis at any stage after reaction with the dithiol addition. In one embodiment, the first and second isotope-coded proteins are combined after dithiol addition and before reaction with the affinity reagent. Mass spectral analysis of the first and second isotope-coded proteins provide quantitative information relating to the difference in the amounts (e.g., expression levels) of the proteins in the first and second protein mixtures.

In another embodiment of the method, first and second affinity-labeled isotope-coded proteins are prepared as described above. These modified proteins are then digested to provide first and second peptide fragments, those fragments that include the dithiol addition product being affinity labeled and isotope coded. The first and second affinity-labeled peptide fragments are isolated on a solid phase to provide first and second isolated affinity-labeled isotope-coded peptide fragments. The first and second isolated peptide fragments are released from the solid phase to provide first and second released peptide fragments. The first and second released peptide fragments are then analyzed. In one embodiment, the first and second isotope-coded peptide fragments are combined and then analyzed by mass spectrometry. The mass spectral analysis of the first and second isotope-coded peptide fragments provide quantitative information relating to the difference in the amounts (e.g., expression levels) of the proteins in the first and second protein mixtures.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a flow diagram illustrating a representative method of the invention for mass spectrometric analysis of isotope-coded peptide fragments;

FIG. 2 is a flow diagram illustrating a representative method of the invention for electrochemiluminescent analysis of biotinylated proteins;

FIG. 3 is a mass spectrum illustrating the products formed by β-elimination of and dithiol addition to representative phosphoserine-containing peptides (KRPSQRHGSKY-NH₂ and RRREEESEEEAA) in accordance with the invention;

FIG. 4 is a mass spectrum illustrating the products formed by β-elimination of and dithiol addition to representative phosphothreonine-containing peptides (KRTIRR and LKRATLG-NH₂) in accordance with the invention;

FIG. 5 is a mass spectrum illustrating the product formed by β-elimination of and dithiol addition to representative N-acetyl glucosamine serine-containing peptide (YSPTSPSK) in accordance with the invention;

FIG. 6 is a mass spectrum illustrating the product formed by β-elimination of and dithiol addition to representative N-acetyl glucosamine threonine-containing peptide (YSPTTPSK) in accordance with the invention;

FIG. 7 is a mass spectrum illustrating the quantitation of representative phosphothreonine-containing peptides (KRTIRR and LKRATLG-NH₂) labeled with dithiothreitol d₀/d₆ (2:1) in accordance with the invention;

FIG. 8 is a mass spectrum illustrating the quantitation of representative phosphoserine-containing peptides (KRPSQRHGSKY-NH₂ and RRREEESEEEAA) labeled with dithiothreitol d₀/d₆ (1:2) in accordance with the invention;

FIG. 9 is a mass spectrum of a casein peptide (QAMEDIK) isolated by biotinylation, released from avidin by dithiothreitol, and alkylated with iodoacetamide in accordance with the invention; and

FIG. 10 is an image of an electrochemiluminescent blot of different concentrations of a representative phosphoprotein (α-casein) labeled in accordance with the invention in a constant concentration of protein mixture (LCA bovine, OVAL Chick, LACB bovine, G3P rabbit, PHS2 rabbit): (a) and (g) 100 ng (α-casein; (b) and (f) 400 ng α-casein; (c) and (e) 200 ng α-casein; and (d) no α-casein.

### DETAILED DESCRIPTION OF THE INVENTION

In one aspect, the invention provides a method for incorporating a stable isotope into a protein or peptide fragment. In the method, a protein or peptide fragment is reacted with an agent that includes one or more stable isotopes to provide an isotope-labeled protein or peptide fragment. The method includes isotope labeling of a protein or a peptide fragment residue derived from an amino acid residue capable of β-elimination toprovide an α,β-unsaturated amino acid residue.

In the method, a stable isotope is incorporated into the protein or peptide fragment through the reaction of an amino acid residue of the protein or peptide fragment with an agent that includes one or more stable isotopes. As noted above, suitable amino acid residues include any residue capable of β-elimination to provide an α,β-unsaturated residue that can further react with the isotope-labeled agent. Residues capable of β-elimination include β-phosphoester-containing residues, such as phosphoserine and phosphothreonine, and N-acetyl glucosamine-containing residues, such as N-acetyl glucosamine serine and N-acetyl glucosamine threonine. In one embodiment, the isotope-labeled protein or peptide fragment includes a residue derived from a phosphoserine residue, a phosphothreonine residue, an N-acetyl glucosamine serine residue, or an N-acetyl glucosamine threonine residue and is labeled with an isotope-labeled dithiol. Peptide fragments can be obtained from proteolytic digestion of a protein.

Base treatment of a protein or peptide fragment capable of β-elimination can provide the α,β-unsaturated residue for reaction with the isotope-labeled agent. Suitable base treaments include treatment with barium hydroxide, lithium hydroxide, sodium hydroxide, potassium hydroxide, and mixtures thereof, among others.

In one embodiment of the method, the isotope-labeled protein or peptide fragment is obtained by reacting the protein or peptide fragment with a base and an isotope-labeled dithiol to provide an isotope-labeled dithiol addition product that is a thioether having an available sulfhydryl group. The sulfhydryl group of the resulting modified protein or peptide fragment can be capped by an alkylating agent, or alternatively, the sulfhydryl group can be reacted with an affinity reagent to provide a protein or peptide fragment that is both affinity- and isotope-labeled. The labeled protein or peptide fragment can be isolated on and subsequently released from a solid phase. In one embodiment, the released isotope-labeled protein or peptide fragment includes a sulfhydryl group that can be alkylated.

As used herein, the term “affinity reagent” refers to a reagent that introduces one member of a specific binding pair into the protein or peptide fragment such that the resulting protein or peptide fragment, referred herein to as an “affinity-labeled” protein or peptide fragment, can be captured and isolated by a solid phase bearing the other member of the specific binding pair. Suitable specific binding pairs are known and include, for example, sugar ligand and lectin, hapten/antigenic determinant ligand and antibody, Fc ligand and protein A, nucleic acid and complementary nucleic acid oligomer, polymer, or analog, among others. In one embodiment, the specific binding pair is a biotin/avidin system. In such a system, the affinity reagent is a biotinylation reagent, the affinity-labeled protein or peptide fragment is a biotinylated protein or peptide fragment, and the solid phase is an avidin solid phase.

A biotinylation reagent useful in the method of the invention includes a biotin moiety covalently attached to a protein reactive moiety such that the protein reactive moiety when coupled to protein provides a cleavable linkage intermediate the biotin moiety and protein reactive moiety. Cleavage of the linkage facilitates the release of the protein or peptide fragment from the solid phase. In one embodiment, the cleavable linkage is a disulfide linkage.

The reagent's biotin moiety can be any one of a variety of biotin derivatives and analogs that are effective in avidin binding. Suitable biotin moieties include those moieties that enable the biotinylated peptide fragment to be isolated by avidin and related avidin proteins. Representative biotin moieties include biotin derivatives such as iminobiotin, biocytin, and caproylamidobiotin, and biotin analogs such as desthiobiotin and biotin sulfone. In one embodiment, the biotin moiety is biotin.

The reagent's protein reactive moiety is a functional group that is reactive with a protein residue's functional group. The reagent's protein reactive moiety can be reactive toward a variety of functional groups including, for example, a sulfhydryl group (cysteine), an amino group (lysine), a hydroxy group (serine), and a carboxy group (glutamic acid). In one embodiment, the reactive moiety is reactive toward a sulfhydryl group. Suitable protein reactive functional groups include acylating groups (e.g., carboxylic acids and their reactive derivatives) and alkylating groups (e.g., α-halo carboxylic acids and their derivatives), among others In one embodiment, the protein reactive moiety is a pyridyldithiol moiety.

In one embodiment, the biotinylation reagent is N-[6-(biotinamido)hexyl]-3′-(2′-pyridyldithio)propionamide, Biotin-HPDP, commercially available from Pierce Chemical Co., Rockville, Ill.

As noted above, in one embodiment of the method, the affinity-labeled protein or peptide fragment is isolated on a solid phase. Suitable solid phases include any solid phase capable of capturing the affinity-labeled protein or peptide fragment. The solid phase bears the complement member of the binding pair. For biotinylated proteins and peptide fragments, the solid phase is an avidin solid phase. As used herein, the term “avidin” refers to any biotin-binding protein other than an immunoglobulin that binds biotin including both natural proteins and recombinant and genetically engineered proteins. The term includes the two common biotin-binding proteins known as “egg white or avian avidin” and “streptavidin.” Egg white or avian avidin, commonly referred to simply as avidin, is a protein that is a constituent of egg white and forms a noncovalent complex with biotin. Streptavidin is a protein isolated from the actinobacterium Streptomyces avidinii and also forms a noncovalent complex with biotin. Other bacterial sources of biotin binding proteins are also known. Both egg white avidin and streptavidin are tetrameric proteins in which the biotin binding sites are arranged in pairs on opposite faces of the avidin molecule. The term also refers to avidin derivatives including succinyl avidin, ferritin avidin, enzyme avidin and crosslinked avidin.

In one embodiment, the isolated protein or peptide fragment is released from the solid phase by cleaving a cleavable linkage, for example, a disulfide linkage, incorporated into the protein or peptide fragment on reaction with the affinity reagent. Release from the solid phase provides a protein or peptide fragment having a sulfhydryl group. In one embodiment, the released protein or peptide fragment is the same as the protein or peptide fragment reacted with the affinity reagent.

The stable isotope is incorporated into the protein or peptide fragment by reaction with an isotope-labeled agent. The protein or peptide fragment that has been modified to include an α,β-unsaturated residue can be reacted with a nucleophilic agent that includes one or more stable isotopes. In one embodiment, the isotope-labeled agent is an isotope-labeled dithiol, for example, dithiothreitol-d₆.

Suitable isotope-labeled agents include nucleophilic agents that are capable of reacting with the protein's or peptide fragment's α,β-unsaturated residue and that include one or more stable isotopes. Suitable nucleophilic agents include thiols, amines, and hydroxy compounds. Because the proteins and peptide fragments are ultimately analyzed by mass spectrometry, preferred isotope-labeled agents include two or more stable isotopes. In one embodiment the agent includes at least two stable isotopes. In another embodiment the agent includes at least three stable isotopes. In a further embodiment the agent includes at least four stable isotopes. In another embodiment the agent includes at least five stable isotopes. In yet another embodiment the agent includes at least six stable isotopes.

Stable isotopes useful in the method include carbon (i.e., ¹³C) and hydrogen (i.e., ²H, deuterium) stable isotopes.

In another aspect of the invention, methods for incorporating a stable isotope into a protein or peptide fragment and methods for measuring protein levels in two or more protein or peptide fragment mixtures are provided. The method for measuring protein levels in two or more protein mixtures includes incorporating a stable isotope into the protein or peptide fragment.

In one method, first and second protein mixtures are treated with an isotope-labeled agent. Suitable isotope-labeled agents include those described above. The first protein mixture is treated with a base and a first dithiol, and the second protein mixture is treated with a base and a second dithiol. The base causes β-elimination for those amino acid residues capable of undergoing such elimination to provide an α,β-unsaturated residue that reacts with the dithiol to provide first and second protein mixtures containing dithiol addition residues. The first dithiol includes one or more stable isotopes and the second dithiol includes no stable isotopes or fewer stable isotopes than the first. The resulting modified proteins in the first and second protein mixtures are reacted with an affinity reagent to provide first and second affinity-labeled protein mixtures. Suitable affinity reagents include those described above. The first and second affinity-labeled proteins are isolated on a solid phase to provide first and second isolated affinity-labeled proteins. The first and second isolated proteins are released from the solid phase to provide first and second released proteins. Release from the solid phase can be through cleavage of a cleavable linkage incorporated into the protein on reaction with the affinity reagent. In one embodiment, the released proteins include sulfhydryl groups, which can be alkylated. The first and second isotope-coded proteins can then analyzed. In one embodiment, the first and second isotope-coded proteins are combined prior to analysis. The proteins can be combined at any stage after incorporation of the stable isotope. The isotope-coded proteins can be analyzed by mass spectrometry. The mass spectral analysis of the first and second isotope-coded proteins provide quantitative information relating to the difference in the amounts (e.g., expression levels) of the proteins in the first and second protein mixtures.

In another method, isotope-coded peptide fragments are prepared and then analyzed. In the method, first and second protein mixtures are treated with an isotope-labeled agent. Suitable isotope-labeled agents include those described above. The first protein mixture is treated with a base and a first dithiol, and the second protein mixture is treated with a base and a second dithiol, to provide first and second protein mixtures containing dithiol addition residues. The first dithiol includes one or more stable isotopes and the second dithiol includes no stable isotopes or fewer stable isotopes than the first. The resulting modified proteins in the first and second protein mixtures are reacted with an affinity reagent to provide first and second affinity-labeled protein mixtures. Suitable affinity reagents include those described above. The first and second affinity-labeled protein mixtures are then digested to provide first and second peptide fragment mixtures, these fragments including first and second affinity-labeled isotope-coded peptide fragments. In one embodiment, the protein mixtures are digested prior to reaction with the affinity reagent. The first and second affinity-labeled peptide fragments are isolated on a solid phase to provide first and second isolated affinity-labeled peptide fragments. The first and second isolated proteins are released from the solid phase to provide first and second released isotope-coded peptide fragments. Release from the solid phase can be through cleavage of a cleavable linkage incorporated into the peptide fragment on reaction with the affinity reagent. In one embodiment, the released peptide fragments include sulfhydryl groups, which can be alkylated. The first and second isotope-coded peptide fragments can be analyzed. In one embodiment, the first and second isotope-coded peptide fragments are combined prior to analysis to provide an isotope-coded peptide fragment mixture. The first and second peptide fragments can be combined at any stage after stable isotope incorporation. The peptide fragment mixture can be analyzed by mass spectrometry. The mass spectral analysis of the first and second isotope-coded peptide fragments provide quantitative information relating to the difference in the amounts (e.g., expression levels) of the proteins in the first and second protein mixtures.

In the above methods, it will be appreciated that the method can include other steps depending on the nature of the protein mixture, information being sought, and the specific analysis. For example, prior to base treatment, the first and second protein mixtures can be reduced with a disulfide reducing agent to produce cysteine residues that can be capped with an alkylating agent to eliminate possible interference of these residues with other method steps. Alternatively, the protein mixtures can be oxidized to prevent interference from cysteinyl residues. As noted above, the protein or peptide fragment mixtures can be combined for further treatment or analysis after the stable isotope has been incorporated into one of the protein or peptide fragment mixtures (i.e., after the protein or peptide fragment has been isotope coded).

It will be appreciated that the method of the invention includes measuring protein levels in two or more protein mixtures. The number of protein mixtures that can be analyzed by the method will depend on the complexity of the protein mixtures and the nature of the isotope-labeled agents. The greater the number of protein mixtures, the greater the number of distinct isotope-labeled agents required for the analysis. Each protein mixture merely requires a distinct isotope-labeled agent. Accordingly, the method of the invention is not limited to comparing protein levels in two protein mixtures, but is applicable to determining the protein levels in a plurality of protein mixtures.

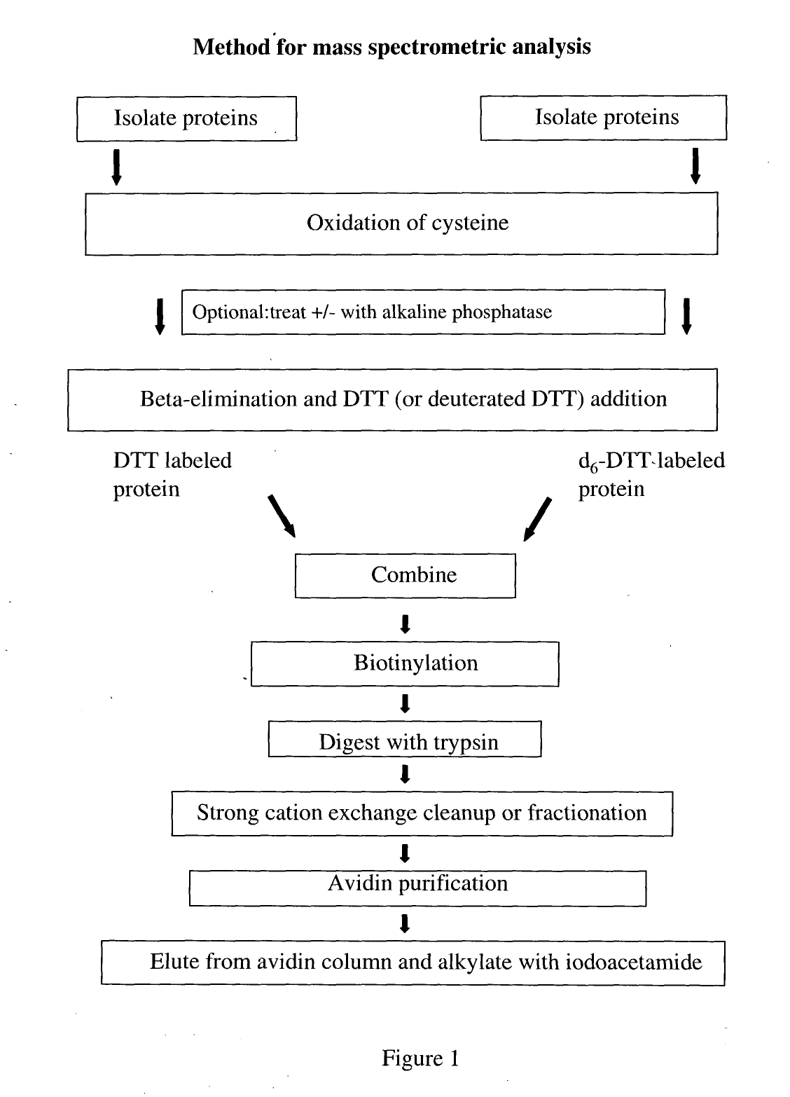

One representative method of the invention is schematically illustrated in FIG. 1. The method is suitable for mass spectrometric analysis of the resulting isotope-coded peptide fragments. Referring to FIG. 1, in the representative method, sample proteins' (e.g., isolated proteins from control cells and isolated proteins from treated cells) cysteine residues are oxidized. Base and isotope-labeled dithiol are then added to the mixtures. The resulting thioether residues having an available sulfhydryl group are biotinylated with a biotinylation reagent having a reducible linker. Next the samples are digested with a protease such as trypsin. The biotinylated peptides are then isolated using avidin beads, and eluted from the avidin with a reducing reagent. The thiols of the released fragments are then alkylated. In one embodiment, the peptide fragments are subjected to cation exchange or fractionation prior to avidin purification.

Estimated yields for elimination and dithiol addition for phosphoserine-containing proteins is greater than about 95 percent; about 75 percent for phosphothreonine-containing proteins; greater than about 95 percent for N-acetyl glucosamine serine-containing proteins; and about 60 percent for N-acetyl glucosamine threonine-containing proteins. The yields for biotinylation and avidin purification are about 70 percent.

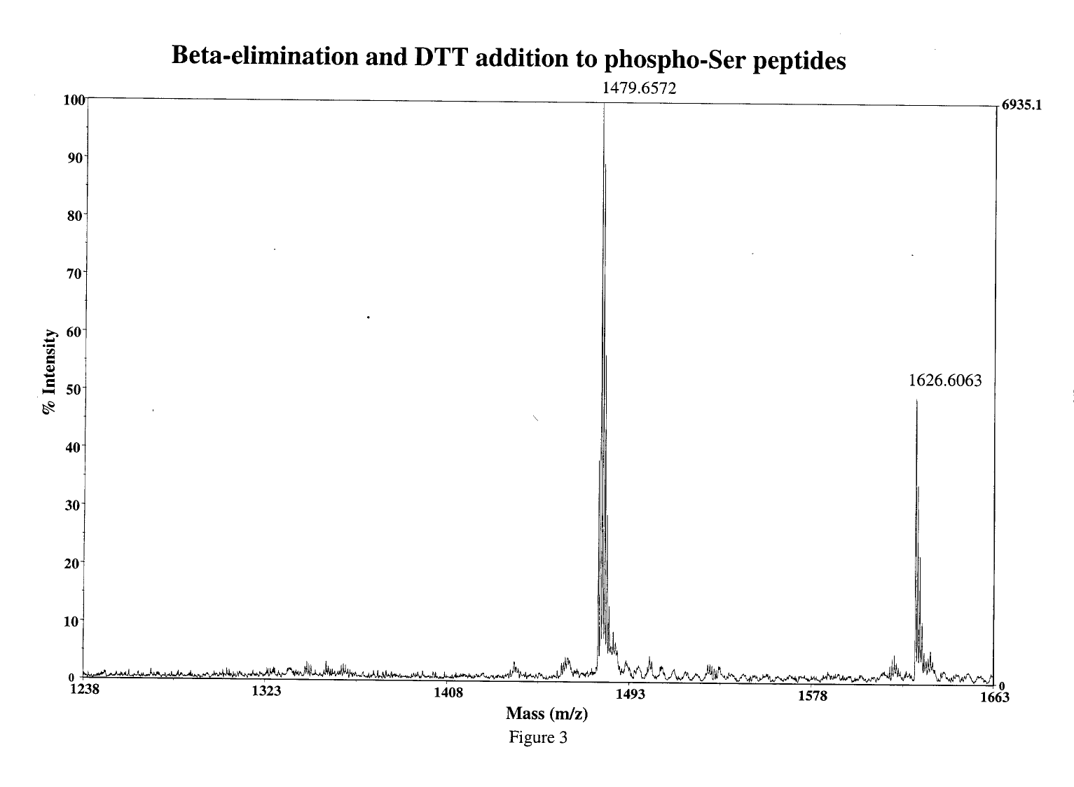

A representative method for preparing a protein sample for mass spectrometric analysis is described in Example 1. Mass spectra obtained for protein samples in accordance with the representative method are presented in FIGS. 3-9.

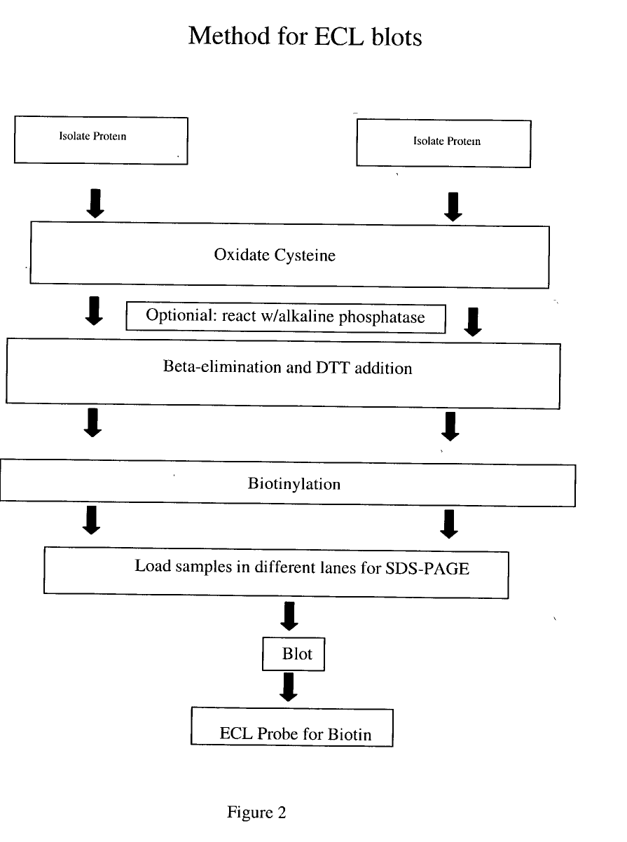

As an alternative to mass spectrometric analysis, Western blots of biotinylated proteins can be used to assess changing levels of phosphorylation and glycosylation using the method of the invention. Accordingly, another representative method of the invention is schematically illustrated in FIG. 2. The method is suitable for electrochemiluminescent (ECL) analysis of the resulting affinity-labeled proteins. Referring to FIG. 2, in the representative method, sample proteins' (e.g., isolated proteins from control cells and isolated proteins from treated cells) cysteine residues are oxidized. Base and dithiol are then added to the mixtures. The resulting thioether residues having an available sulfhydryl group are biotinylated with a biotinylation reagent. In one embodiment, the biotinylation agent is biotin-BMCC (1-biotinamido-4-[4′-(maleimidomethyl)cyclohexane carboxarnido]butane; Pierce, Rockford, Ill.). The biotinylated proteins are then loaded as samples in different lanes for SDS-PAGE. After electrophoresis, the gel is blotted and the blot probed for biotin by an ECL probe.

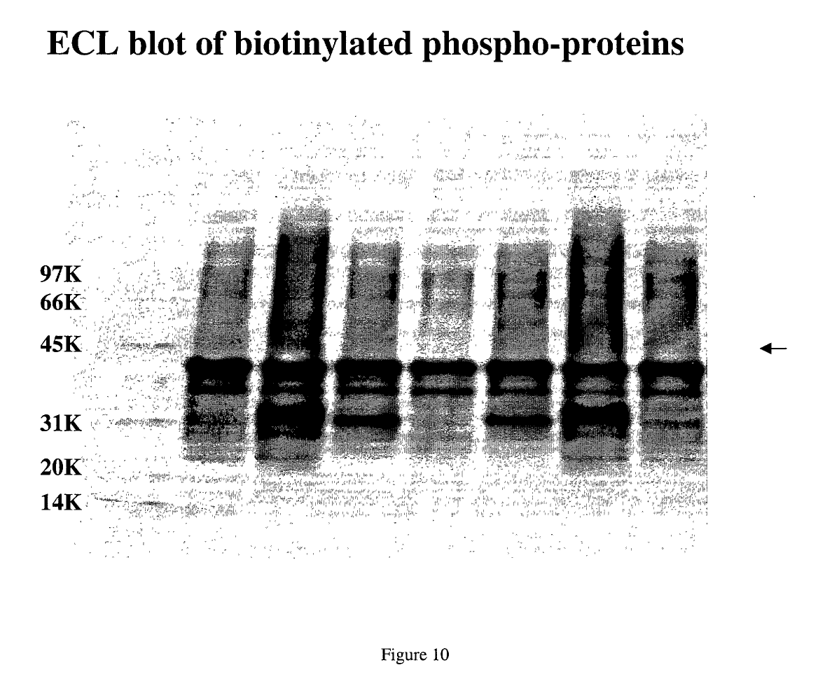

A representative method for preparing a protein sample for Western blot spectrometric analysis is described in Example 2. An image of an electrophoretic gel obtained for protein samples in accordance with the representative method are presented in FIG. 10.

To summarize, in one aspect of the invention, a method for making quantitative protein measurements is provided. The method can be used for comparing changes in protein expression between two or more protein mixtures. The method includes incorporating a stable isotope into a protein or peptide fragment as described above. Mass spectral analysis of the isotope-coded protein or peptide fragments obtained from the protein mixtures provides quantitative information relating to the level of expression of the proteins in each mixture. The method can be used to identify and quantitate protein or peptide fragments that include residues capable of β-elimination to provide an α,β-unsaturated residue of the protein or peptide fragment that can further react with an isotope-labeled dithiol. Thus, the method can be used to identify and quantitate phosphoserine-containing proteins and peptide fragments, phosphothreonine-containing proteins and peptide fragments, N-acetyl glucosamine serine-containing proteins and peptide fragments, and N-acetyl glucosamine threonine-containing proteins and peptide fragments. The modification sites and relative changes in the levels of modification can be identified by the method.

The following examples are offered by way of illustrating, not limiting, the invention.

### EXAMPLES

### Example 1

Representative Quantitative Protein Measurement: Mass Spectrometric Analysis

In this example, a representative method for preparing a protein sample for mass spectrometric analysis in accordance with the invention is described. A flow diagram illustrating the representative method is illustrated in FIG. 1. The sample for analysis is prepared as follows.

Oxidation of protein sample using performic acid. Performic acid is prepared by mixing 0.1 ml 30 hydrogen peroxide with 0.9 ml 98-100% formic acid, and allowing the mixture to react for at least one hour at room temperature. The performic acid reactant is then cooled on ice before adding 50 ul of the reactant to 50 ug protein. The protein oxidation proceeds for three hours on ice. The reaction is stopped by dilution with 450 ul (10×) ice-cold water, and vacuum centifuged dry.

β-elimination and dithiothreitol (DTT) addition. 50 ug of dried oxidized protein is solubilized in 50 ul 6 M guanidine hydrochloride containing 400 mM DTT (or d₁₀-DTT [Aldrich or Cambridge Isotopes]) plus 800 mM lithium hydroxide (LiOH) (to neutralize the relatively acidic thiols in DTT). 100 ul of saturated barium hydroxide (Ba(OH)₂) (approximately 150 mM) is added and incubated at room temperature for three hours under nitrogen. For Western blot experiments (see Example 2 below), the reaction time can be shortened to about 30 minutes depending on the sensitivity required. Note that four of the ten deuterium atoms in d₁₀-DTT are rapidly exchanged with protons in the aqueous solution, and only six are in a stable deuterium-carbon bond. Hence, although the reactant is d₁₀-DTT, the mass difference observed between DTT and d₁₀-DTT labeled peptides is only 6 mass units (u). The reaction is stopped by the acidification with 10% trifluoroacetic acid (TFA).

If two samples are being compared for relative quantitative measurements (differentially labeled with DTT and d₁₀-DTT), the two samples are combined at this point. Excess DTT is removed by protein precipitation by the addition of 400 ul methanol, 100 ul chloroform, and 300 ul water. The mixture is vortexed and centrifuged at 10,000 rpm for three minutes. The upper phase is removed and discarded; care is taken not to touch the meniscus where the protein resides. 400 ul of ice-cold methanol is added, vortexed, and allowed to sit for 2-16 hours on ice. The precipitated protein is pelleted by centrifugation at 14,000 rpm for 10 minutes at 4° C. The supernatant is removed and discarded, and the protein pellet is briefly dried by vacuum centrifugation.

HPDP biotinylation. Following performic acid oxidation, β-elimination, and DTT addition, the modified and precipitated protein is solubilized in 80 ul 8 M urea (stored over AG 501-X8 mixed bed resin), followed by the addition of 80 ul 2 mM EDTA and 100 mM Tris pH 8. The solubilized protein is biotinylated by adding 10 ul 4 mM HPDP-biotin (Pierce Chemical Co.) in dry DMSO for 90 minutes at room temperature in the dark.

Trypsin digestion. Following the biotinylation reaction, excess biotin can be removed using a second precipitation step, as described above. The precipitated proteins are then solubilized in 10 ul 8 M urea and then the urea is diluted using 30 ul 50 mM Tris pH 8. Trypsin is added at a substrate to enzyme ration of 50-100:1 and digested 4-16 hours at 37° C. To avoid tryptic digestion of the avidin beads (the next step), the trypsin is treated with tosyl lysine chloromethyl ketone (TLCK) at a concentration of 50 ug/ml for 60 minutes at 37° C. The TLCK treated sample is boiled for 20 minutes, and the pH is dropped to 5 using 3 M sodium acetate. Most of the tryptic activity is eliminated by TLCK and boiling, and any residual activity is greatly inhibited by the lowered pH.

As an alternative to using a second precipitation step, the mixture containing excess biotinylation reagent can be digested directly with trypsin, and the excess reagent and trypsin is removed from the tryptic peptides using a strong cation exchange column. The 4 M urea solution is diluted to 2 M using water and trypsin is added at a substrate to ratio of 100:1, and the proteins are digested 4-16 hours at 37° C. The Tris buffer salts are diluted, and the pH is dropped by diluting the sample with two volumes of 10 mM sodium phosphate pH 3 prior to loading on the strong cation exchange column. The column is washed with 10 mM sodium phosphate pH 3, and the bound peptides are eluted using 10 mM sodium phosphate pH 3 containing 500 mM KCl.

Avidin isolation of biotinylated peptides. 100-200 ul of a 50% slurry of monoavidin beads (Pierce) is placed in a BioRad mini column, washed twice with 1 ml 30% acetonitrile containing 0.4% TFA. The beads are then washed twice with 1 ml PBS pH 7.2, followed by three washes of 1 ml 0.15 sodium acetate 0.15 M NaCl pH 5.5. The tryptic peptides are added to the washed beads and incubated for 20 minutes with gentle vortexing at room temperature. The column is drained, and washed once with 1 ml 0.15 sodium acetate 0.15 M NaCl pH 5.5. Next the beads are washed three times with 1 ml of 0.1 M Tris pH 8 containing 1 mM EDTA. The biotinylated peptides are eluted from the column by incubating the beads with 100 ul 0.1 M Tris pH 8 containing 1 mM EDTA and 10 mM DTT for 30 minutes at 37° C. The released peptides are recovered and alkylated with 55 mM iodoacetamide for 30 minutes in the dark at room temperature. Excess iodoacetamide is quenched by the addition of 1 ul mercaptoethanol, and the sample is ready for mass spectrometric analysis.

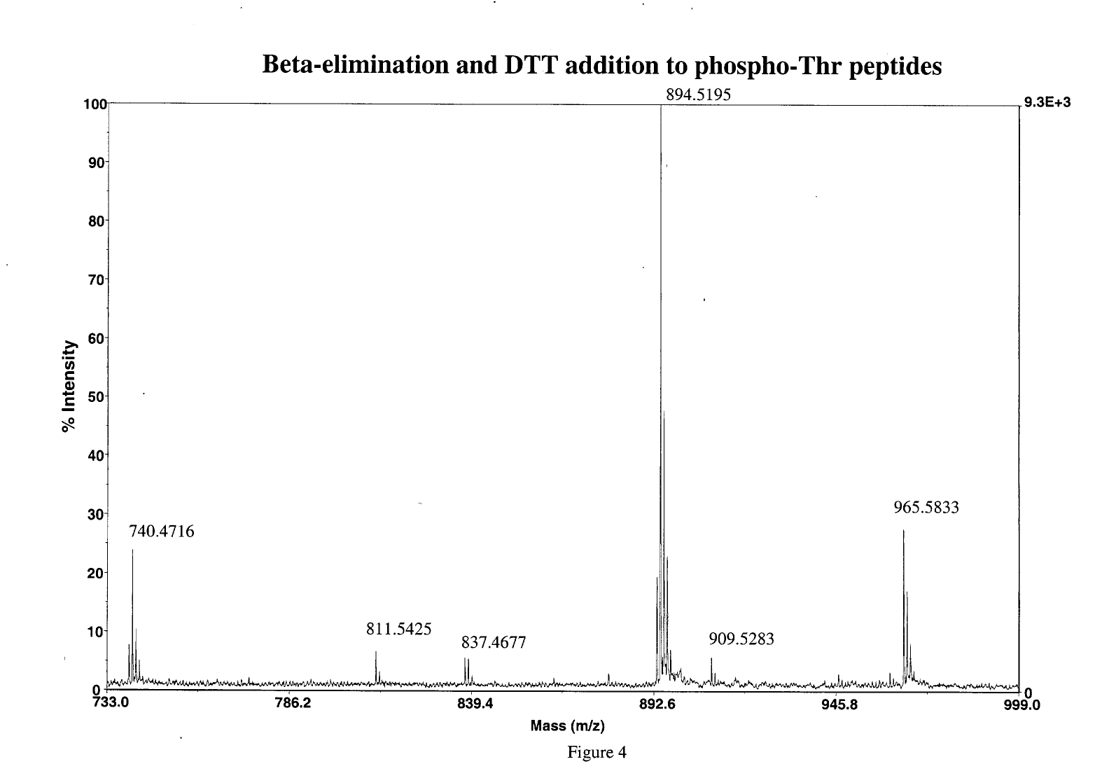

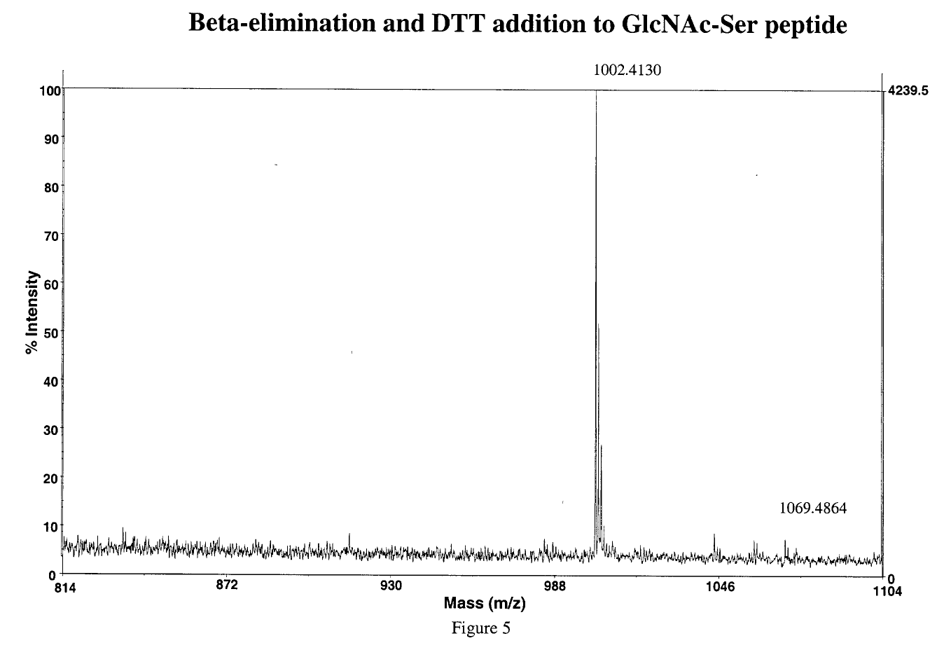

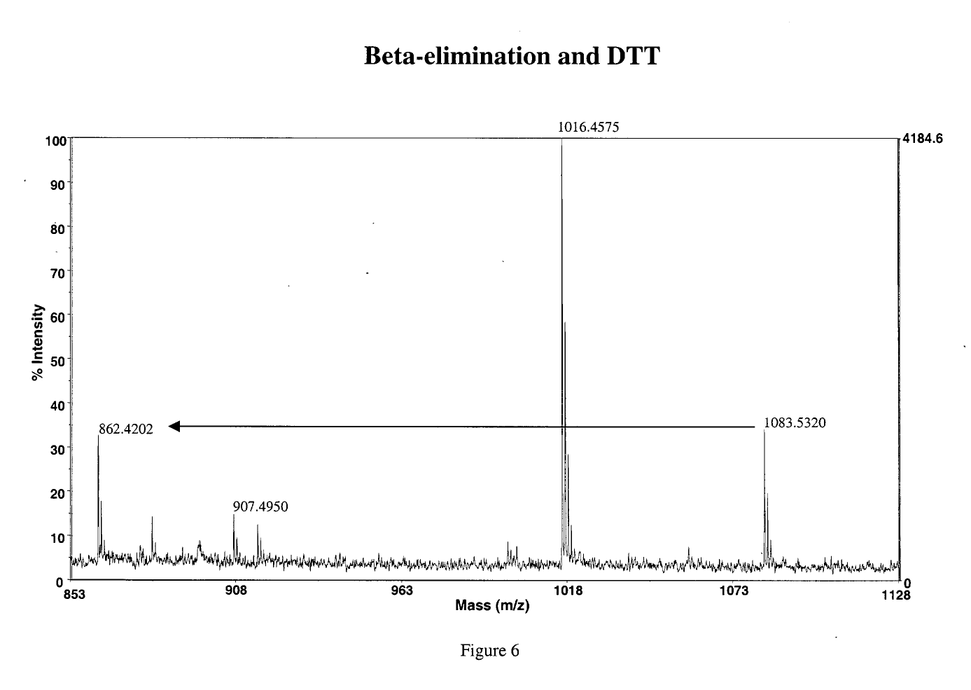

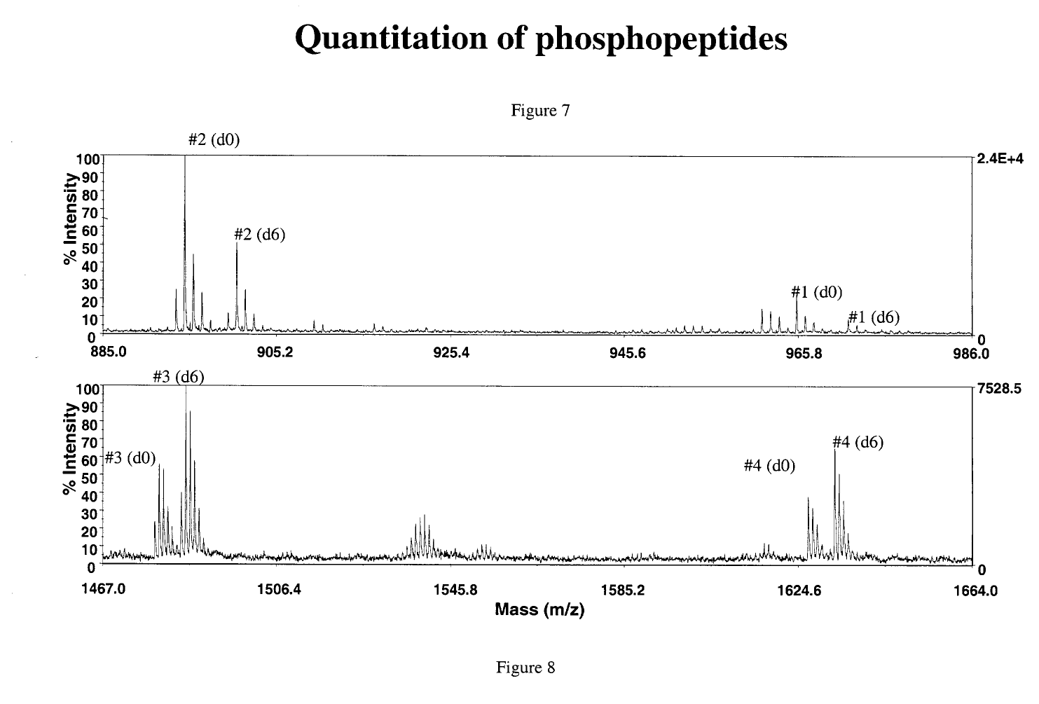

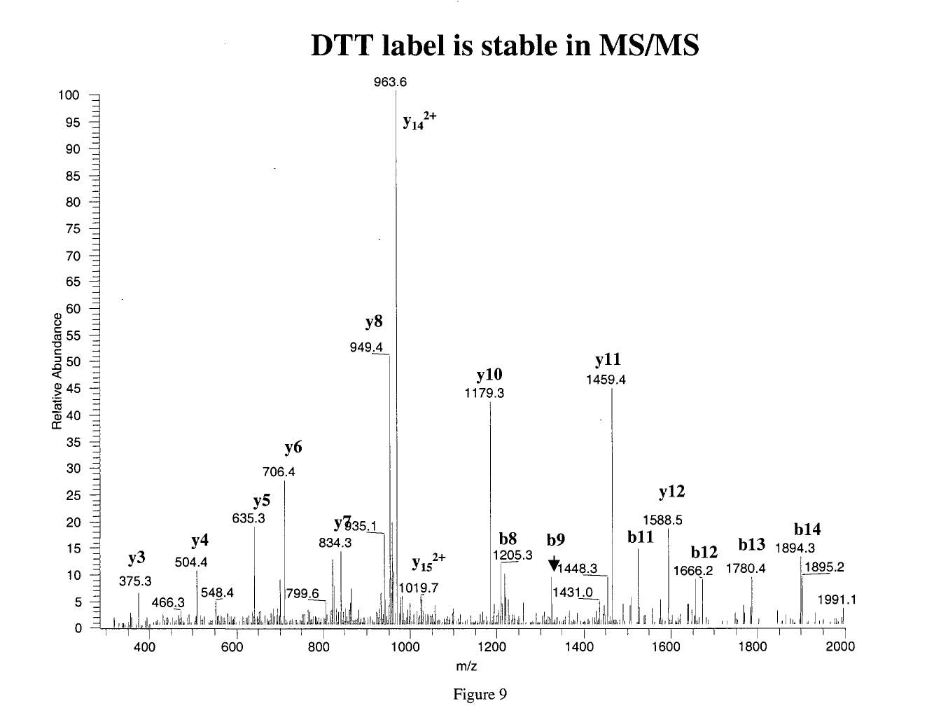

Representative mass spectra obtained for protein samples in accordance with the present invention are presented in FIGS. 3-9. FIG. 3 is a mass spectrum illustrating the products formed by β-elimination of and dithiol addition to representative phosphoserine-containing peptides (KRPSQRHGSKY-NH₂ and RRREEESEEEAA). FIG. 4 is a mass spectrum illustrating the products formed by β-elimination of and dithiol addition to representative phosphothreonine-containing peptides (KRTIRR and LKRATLG-NH₂). FIG. 5 is a mass spectrum illustrating the product formed by β-elimination of and dithiol addition to representative N-acetyl glucosamine serine-containing peptide (YSPTSPSK). FIG. 6 is a mass spectrum illustrating the product formed by β-elimination of and dithiol addition to representative N-acetyl glucosamine threonine-containing peptide (YSPTTPSK). FIG. 7 is a mass spectrum illustrating the quantitation of representative phosphothreonine-containing peptides (KRTIRR and LKRATLG-NH₂) labeled with dithiothreitol d₀/d₆ (2:1). FIG. 8 is a mass spectrum illustrating the quantitation of representative phosphoserine-containing peptides (KRPSQRHGSKY-NH₂ and RRREEESEEEAA) labeled with dithiothreitol d₀/d₆ (1:2). FIG. 9 is a mass spectrum of a casein peptide (QAMEDIK) isolated by biotinylation, released from avidin by dithiothreitol, and alkylated with iodoacetamide.

### Example 2

Representative Quantitative Protein Measurement: Western Blot Analysis

In this example, a representative method for preparing a protein sample for Western blot analysis in accordance with the invention is described. A flow diagram illustrating the representative method is illustrated in FIG. 2. The sample for analysis is prepared as follows.

PEO-maleimide activated biotin. Following performic acid oxidation, beta-elimination, and DTT addition as described above in Example 1, the modified and precipitated protein is solubilized in 50 ul 8 M urea followed by 50 ul of 2 mM EDTA and 100 mM Tris pH 7.2, and biotinlyated for 2 hours at room temperature by the addition of 10 ul of 10 mM PEO-maleimide biotin (biotinyl-3-maleimidopropionamidyl-3,6-dioxaoctanediamine; Pierce, Rockford, Ill.) in PBS. Excess biotin is removed by protein precipitation as described above.

Western blot. Biotinylated protein samples are solubilized in SDS-PAGE reducing sample buffer and loaded onto separate lanes of a SDS-PAGE gel. Following electrophoresis, the gel is washed three times for 30 minutes each in 25 mM Tris, pH 8.3/192 mM glycine/20% methanol prior to electroblotting onto NC membrane. The membrane is blocked in 3% BSA/0.1% Tween/Tris buffered saline (TBS) at room temperature for one hour, and washed in 0.1% Tween/TBS once for 15 minutes and two more times for five minutes. The blot is probed with Streptavidin-HRP prior to ECL imaging.

A representative image obtained for a protein sample in accordance with the invention is presented in FIG. 10. FIG. 10 is an image of an electrochemiluminescent blot of different concentrations of a representative phosphoprotein (α-casein) labeled as described above in a constant concentration of protein mixture (LCA bovine, OVAL Chick, LACB bovine, G3P rabbit, PHS2 rabbit): (a) and (g) 100 ng α-casein; (b) and (f) 400 ng α-casein; (c) and (e) 200 ng α-casein; and (d) no α-casein.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for incorporating a stable isotope into a protein or peptide fragment, comprising:
(a) reacting a protein or peptide fragment having an α,β-unsaturated amino acid residue with a dithiol to provide a dithiol addition product, wherein the dithiol includes one or more stable isotopes, and wherein the dithiol addition product includes a sulfhydryl group; and
(b) reacting the dithiol addition product with an affinity reagent to provide an affinity-labeled protein or peptide fragment that includes one or more stable isotopes.

2. The method of claim 1, wherein the α,β-unsaturated amino acid residue is derived from at least one of a phosphoserine residue, a phosphothreonine residue, an N-acetyl glucosamine serine residue, and an N-acetyl glucosamine threonine residue.

3. The method of claim 1, wherein the stable isotope is at least one of ¹³C and ²H.

4. The method of claim 1, wherein the dithiol comprises d₆-dithiothreitol.

5. The method of claim 1, wherein the affinity reagent comprises a biotinylation reagent.

6. The method of claim 1, wherein the affinity reagent is N-[6-(biotinamido)hexyl]-3′-(2′-pyridyldithio)propionamide.

7. The method of claim 1 further comprising isolating the affinity-labeled protein or peptide fragment on a solid phase, and releasing the protein or peptide fragment from the solid phase.

8. A method for incorporating a stable isotope into a peptide fragment, comprising:
(a) reacting an α,β-unsaturated amino acid residue in a protein mixture with a dithiol to provide a dithiol addition product, wherein the dithiol includes one or more stable isotopes, and wherein the dithiol addition product includes a sulfhydryl group;
(b) reacting the dithiol addition product with an affinity reagent to provide an affinity-labeled protein mixture; and
(c) digesting the affinity-labeled protein mixture to provide affinity-labeled peptide fragments, wherein the peptide fragments comprise affinity-labeled peptide fragments.

9. The method of claim 8, wherein the α,β-unsaturated amino acid residue is derived from at least one of a phosphoserine residue, a phosphothreonine residue, an N-acetyl glucosamine serine residue, and an N-acetyl glucosamine threonine residue.

10. The method of claim 8, wherein the dithiol comprises at least two stable isotopes.

11. The method of claim 8, wherein the stable isotope is at least one of ¹³C and ²H.

12. The method of claim 8, wherein the dithiol comprises d₆-dithiothreitol.

13. The method of claim 8, wherein the affinity reagent comprises a biotinylation reagent.

14. The method of claim 8, wherein the affinity reagent is N-[6(biotinamido)hexyl]-3′-(2′-pyridyldithio)propionamide.

15. The method of claim 8 further comprising isolating the affinity-labeled protein or peptide fragment on a solid phase, and releasing the protein or peptide fragment from the solid phase.

16. A method for measuring protein levels in two protein mixtures, comprising:
(a) reacting an α,β-unsaturated amino acid residue in a first protein mixture with a first dithiol to provide a first dithiol addition product, wherein the first dithiol includes one or more stable isotopes, and wherein the first dithiol addition product includes a sulfhydryl group;
(b) reacting an α,β-unsaturated amino acid residue in a second protein mixture with a second dithiol to provide a second dithiol addition product, wherein the second dithiol is the first dithiol with no stable isotopes or fewer stable isotopes than the first dithiol, and wherein the second dithiol addition product includes a sulfhydryl group;
(c) reacting the first and second dithiol addition products with an affinity reagent to provide first and second affinity-labeled proteins;
(d) isolating the first and second affinity-labeled proteins on a solid phase;
(e) releasing the isolated first and second affinity-labeled proteins from the solid phase; and
(f) analyzing the first and second released proteins.

17. The method of claim 16, wherein the α,β-unsaturated amino acid residue is derived from at least one of a phosphoserine residue, a phosphothreonine residue, an N-acetyl glucosamine serine residue, and an N-acetyl glucosamine threonine residue.

18. The method of claim 16, wherein the first dithiol comprises d₆-dithiothreitol.

19. The method of claim 16, wherein the second dithiol comprises no stable isotopes.

20. The method of claim 16, wherein the second dithiol comprises dithiothreitol.

21. The method of claim 16, wherein the stable isotope is at least one of ¹³C and ²H.

22. The method of claim 16, wherein the affinity reagent comprises a biotinylation reagent.

23. The method of claim 16, wherein the affinity reagent is N-[6-(biotinamido)hexyl]-3′-(2′-pyridyldithio)propionamide.

24. The method of claim 16 further comprising combining the first and second dithiol addition products prior to reacting with the affinity reagent.

25. A method for measuring protein levels in two protein mixtures, comprising:
(a) reacting an α,β-unsaturated amino acid residue in a first protein mixture with a first dithiol to provide a first dithiol addition product, wherein the first dithiol includes one or more stable isotopes, and wherein the first dithiol addition product includes a sulfhydryl group;
(b) reacting an α,β-unsaturated amino acid residue in a second protein mixture with a second dithiol to provide a second dithiol addition product, wherein the second dithiol is the first dithiol with no stable isotopes or fewer stable isotopes than the first dithiol, and wherein the second dithiol addition product includes a sulfhydryl group;
(c) reacting the first and second dithiol addition products with an affinity reagent to provide first and second affinity-labeled proteins;
(d) digesting the first and second affinity-labeled protein mixtures to provide first and second affinity-labeled peptide fragments, wherein the peptide fragments comprise affinity-labeled peptide fragments;
(e) isolating the first and second affinity-labeled peptide fragments on a solid phase;
(f) releasing the isolated first and second affinity-labeled peptide fragments from the solid phase; and
(g) analyzing the first and second released peptide fragments.

26. The method of claim 25, wherein the α,β-unsaturated amino acid residue is derived from at least one of a phosphoserine residue, a phosphothreonine residue, an N-acetyl glucosamine serine residue, and an N-acetyl glucosamine threonine residue.

27. The method of claim 25, wherein the first dithiol comprises d₆-dithiothreitol.

28. The method of claim 25, wherein the second dithiol comprises no stable isotopes.

29. The method of claim 25, wherein the second dithiol comprises dithiothreitol.

30. The method of claim 25, wherein the stable isotope is at least one of ¹³C and ²H.

31. The method of claim 25, wherein the affinity reagent comprises a biotinylation reagent.

32. The method of claim 25, wherein the affinity reagent is N-[6-(biotinamido)hexyl]-3′-(2′-pyridyldithio)propionamide.

33. The method of claim 25 further comprising combining the first and second dithiol addition products prior to reacting with the affinity reagent.

34. A method for measuring protein levels in two protein mixtures, comprising:
(a) oxidizing two protein mixtures to provide a first and second oxidized protein mixtures;
(b) reacting an α,β-unsaturated amino acid residue in the first protein mixture with a first dithiol to provide a first dithiol addition product, wherein the first dithiol includes one or more stable isotopes, and wherein the first dithiol addition product includes a sulfhydryl group;
(c) reacting an α,β-unsaturated amino acid residue in the second protein mixture with a second dithiol to provide a second dithiol addition product, wherein the second dithiol is the first dithiol with no stable isotopes or fewer stable isotopes than the first dithiol, and wherein the second dithiol addition product includes a sulfhydryl group;
(d) combining the first and second dithiol addition products;
(e) reacting the first and second dithiol addition products with a biotinylation reagent to provide first and second biotinylated proteins, wherein the biotinylation reagent is covalently coupled to the dithiol addition residue by a cleavable linkage;
(f) digesting the first and second biotinylated protein mixtures to provide first and second biotinylated peptide fragments, wherein the peptide fragments comprise biotinylated peptide fragments;
(g) isolating the first and second biotinylated peptide fragments on a solid phase;
(h) releasing the isolated first and second peptide fragments from the solid phase; and
(i) analyzing the first and second released peptide fragments.

35. The method of claim 34, wherein the α,β-unsaturated amino acid residue is derived from at least one of a phosphoserine residue, a phosphothreonine residue, an N-acetyl glucosamine serine residue, and an N-acetyl glucosamine threonine residue.

36. The method of claim 34, wherein the first dithiol comprises d₆-dithiothreitol.

37. The method of claim 34, wherein the second dithiol comprises dithiothreitol.

38. The method of claim 34, wherein the biotinylation reagent comprises a sulfhydryl reactive moiety.

39. The method of claim 34, wherein the biotinylation reagent comprises a cleavable linkage between a biotin moiety and a sulfhydryl reactive moiety.

40. The method of claim 34, wherein the biotinylation reagent is N-[6-(biotinamido)hexyl]-3′-(2′-pyridyldithio)propionamide.

41. The method of claim 34, wherein releasing the isolated peptide fragments comprises treating with dithiothreitol.

42. A method for identifying a protein or peptide fragment, comprising:
(a) reacting an α,β-unsaturated amino acid residue in a protein mixture with a dithiol to provide a dithiol addition product, wherein the dithiol addition product includes a sulfhydryl group;
(b) reacting the dithiol addition product with an affinity reagent to provide an affinity-labeled protein or peptide fragment mixture;
(c) separating the affinity-labeled protein or peptide fragment mixture by electrophoresis to provide separated affinity-labeled proteins or peptide fragments;
(d) treating the affinity-labeled proteins or peptide fragments with a reporting agent that is a specific binding partner to the affinity-labeled protein or peptide fragment.
(e) The method of claim 76, wherein the α,β-unsaturated amino acid residue is derived from at least one of a phosphoserine residue, a phosphothreonine residue, an N-acetyl glucosamine serine residue, and an N-acetyl glucosamine threonine residue

43. The method of claim 42, wherein the dithiol comprises dithiothreitol.

44. The method of claim 42, wherein the affinity reagent comprises a biotinylation reagent.

45. The method of claim 42, wherein the affinity reagent is N-[6-(biotinamido)hexyl]-3′-(2′-pyridyldithio)propionamide.
